(19) United States
(12) Patent Application Publication
(10) Pub. No.: US 2025/0346543 A1
(43) Pub. Date: Nov. 13, 2025

(54) LPG SYNTHESIS FROM BIO-BASED SOURCES

(71) Applicant: Lowell Street Ventures LLC, Washington, DC (US)

(72) Inventors: Dennis John O’REAR, Petaluma, CA (US); Paul Frank Bryan, Pinole, CA (US); Steven Francis Sciamanna, Orinda, CA (US)

(73) Assignee: Lowell Street Ventures LLC, Washington, DC (US)

(21) Appl. No.: 18/661,268

(22) Filed: May 10, 2024

(51) Int. Cl. C07C 1/04; C07C 7/13

(52) U.S. Cl. CPC C07C 1/044; C07C 1/043; C07C 1/0435; C07C 7/13; C07C 2523/06; C07C 2523/26; C07C 2523/42; C07C 2523/44; C07C 2523/46; C07C 2523/72; C07C 2523/745; C07C 2529/70

(57) ABSTRACT

A method is provided for synthesizing bio-based LPG from renewable sources via a bio-based synthetic gas feedstock. The method includes treating unsaturated hydrocarbons that are generated in the synthesis reactions. Removing the unsaturated hydrocarbons from a recycle stream comprising unreacted synthesis gas components serves to improve catalyst life and activity of the LPG synthesis catalysts.

### FIELD OF THE DISCLOSURE

The invention is related to synthesis of liquefied petroleum gas (LPG) from bio-based sources.

### BACKGROUND

The issue of climate change is a real and growing problem. Carbon dioxide emissions from the burning of fossil fuels are a significant driving force. To address this issue various government regulations will soon require the use of renewable fuels as a component in conventional fuels, including propane and LPG. For example, the European renewable energy policy framework has just raised the EU target share of renewables to 40% by 2030.

The California Air Resources Board regulations require transportation fuel producers and importers to meet specified average carbon intensity requirements for fuel. Low Carbon Fuel Standard regulated fuels include natural gas, electricity, hydrogen, gasoline mixed with at least 10% corn-derived ethanol, biomass-based diesel, and propane.

Inland countries in Africa face a different issue. Propane and LPG are widely used as cooking fuels, but they must be imported and transported over land. This significantly increases their costs. Production of propane and LPG near the inland markets would be a cost savings, and, when produced from renewable resources, would also address climate change issues.

Thus, the demand for propane and LPG made from a non-fossil and/or renewable resources (bio-based propane and bio-based LPG) is real, current, and worldwide.

There are several other issues regarding the synthesis/conversion reaction sequence using synthesis gas as a reactant. In one, significant amounts of hydrogen are present in the exit gas from the reactor. While recycling has been proposed, conventional recycling processes involve recompression of the recycled hydrogen, at significant energy costs. Secondly, in conventional processing, over 10% of the carbon introduced as reaction feedstock is produced as carbon dioxide. This represents a waste of the valuable carbon monoxide resource in the bio-based synthesis gas. Accordingly, there continues to be a need for producing LPG from bio-based sources, using a sequence of processing steps that result in high LPG yields at low loss of the synthetic gas components, H₂, CO, and CO₂.

### SUMMARY OF THE DISCLOSURE

In one aspect, the present disclosure describes various embodiments of a system and a process for converting bio-based synthesis gas comprising CO and H₂ into LPG. One source of the bio-based synthesis gas is light hydrocarbon gases, principally methane, that have been generated from and recovered from one or more biomass sources.

In another aspect, the present disclosure provides an improved process for converting synthesis gas to light (i.e., C3+) hydrocarbons, principally LPG, that has multiple uses as a biofuel source of power and heat.

In another aspect, the present disclosure provides an improved process for converting greenhouse gases, principally methane, into bio-based fuels that may be used as automotive, commercial, and domestic sources of heat and power with reduced, and in some cases, minimal environmental impact.

In another aspect, the present disclosure provides a process for converting hydrocarbon gases generated from agricultural and municipal sources, including wastewater and sewage treating and solids disposal sites, into low environmental impact fuels.

In another aspect, the present disclosure provides an improved process for producing LPG from a synthesis process while recovering and efficiently recycling the unreacted synthesis gas components. The improved recycling process removes components from the recycle, principally unsaturated hydrocarbons, that may have a detrimental effect on catalyst activity and catalyst life.

In another aspect, the present disclosure is directed to a method for producing bio-based LPG, comprising: a) synthesizing an LPG-enriched gaseous effluent from a blended bio-based synthesis gas in one or more catalytic reaction zones, wherein the blended bio-based synthesis gas comprises a treated synthesis gas recycle stream and a fresh bio-based synthesis gas, and wherein the LPG-enriched gaseous effluent contains unsaturated hydrocarbons; b) separating the LPG-enriched gaseous effluent into a synthesis gas recycle stream and at least one LPG-enriched hydrocarbon product; c) removing at least a portion of the unsaturated hydrocarbons contained in either the LPG-enriched gaseous effluent or the synthesis gas recycle stream, or both, in one or more treatment steps, and producing the treated synthesis gas recycle stream containing less than 5 mol % unsaturated hydrocarbons, based on the total moles of treated synthesis gas recycle stream that is recycled to the oxygenate synthesis zone; and d) blending at least a portion of the treated synthesis gas recycle stream with the fresh bio-based synthesis gas and forming the blended bio-based synthesis gas of step a).

The fresh bio-based synthesis gas is prepared by contacting a biogas comprising biomethane with an oxidizing gas selected from O₂, CO₂ and H₂O or combinations thereof at reforming reaction conditions in a reforming reaction zone.

The method of synthesizing an LPG-enriched gaseous effluent may include a two-step reaction process, including a) reacting the blended bio-based synthesis gas in an oxygenate synthesis zone containing an oxygenate synthesis catalyst and forming an oxygenated reaction product comprising oxygenates and unreacted bio-based synthesis gas, wherein the oxygenates include at least 50 mol % methanol; and b) reacting at least a portion of the oxygenated reaction product in an oxygenate conversion zone containing an oxygenate conversion catalyst and forming the LPG-enriched gaseous effluent.

The unsaturated hydrocarbons that may affect the performance of LPG synthesis catalysts include olefins and aromatics that may be generated by one of the steps of the LPG synthesis reactions. Typical unsaturated hydrocarbons that may affect catalyst performance include, for example, ethylene, propylene, butene, pentene, benzene, toluene and the like. These unsaturated hydrocarbons may be present in the bio-based LPG in amounts as high as 6 mol % or higher. Unsaturated hydrocarbons that remain in the synthesis gas recycle may be removed by hydrogenation, by adsorption on a solid adsorbent, or by absorption in a liquid, resulting in an unsaturated hydrocarbons content of the treated synthesis gas recycle stream of less than 5 mol %, or less than 2 mol %, or less than 1 mol %, or less than 0.1 mol %, or even less than 0.01 mol % of the treated synthesis gas recycle stream.

In effect, the disclosed embodiments of the present invention enable preparation of LPG from bio-based sources at high LPG yield and low loss of synthetic gas components H₂, CO, and CO₂ in the recycle process.

### BRIEF DESCRIPTION OF THE DRAWINGS

FIG. 1 illustrates an embodiment of the present invention describing a method for producing bio-based LPG.

FIG. 2 illustrates another embodiment of the present invention describing a method for synthesizing an LPG-enriched effluent.

FIG. 3 illustrates a schematic drawing of a system and a process for synthesizing a bio-based LPG from syngas that is derived from a bio-based source.

### DETAILED DESCRIPTION

As used herein, “C2− hydrocarbons” refers to hydrocarbons composed of 1 or 2 carbon atoms (e.g., methane or ethane), either alone or in combination. Likewise, “C2+ hydrocarbons” refers to hydrocarbons composed of 2 or more carbon atoms (e.g., ethane, propane, etc.). Likewise, C3 hydrocarbons refers to hydrocarbons composed of 3 carbon atoms (e.g., propane). Likewise, C4 hydrocarbons refers to hydrocarbons composed of 4 carbon atoms (e.g., butane). Likewise, C4− hydrocarbons refers to hydrocarbons composed of 1 to 4 carbon atoms (e.g., methane, ethane, propane, and butane). Likewise, “C5+ hydrocarbons” refers to hydrocarbons composed of five or more carbon atoms (pentane, hexane, etc.).

As used herein, the term “LPG” refers to liquefied petroleum gas, a composition comprising a mixture of hydrocarbon gases, such as propane, propylene, butylene, isobutane, or n-butane. The term may refer to slightly different compositions, depending on the market to which the LPG is directed. For example, European LPG is a mixture of light hydrocarbons comprising propane and optionally n-butane and iso-butane. It is synonymous with AutoGas. Smaller amounts of ethane and C5+ may be present. In the United States, LPG mostly refers to propane. Bio-based LPG is LPG made from biogas.

As used herein, the terms “LPG and “bio-based LPG” are used interchangeably unless otherwise specified. According to the present disclosure, the composition of any LPG produced as described herein may be tailored by distillate fractionation; and the present process is suitable for producing LPG across a range of compositions. Thus, the term “LPG” as used herein refers to a mixture of propane and butane (n-butane and/or i-butane) in any composition ratio.

As used herein, “H₂”, “CO”, “CO₂”, “MeOH”, and “DME” have conventional designations, referring to molecular hydrogen, carbon monoxide, carbon dioxide, methanol, and dimethyl ether.

As used herein, a “bio-based” material refers to a material that is sourced from one or more natural resources, which will replenish to replace the portion depleted by usage and consumption, either through natural reproduction or other recurring processes in a finite amount of time in a human time scale.

As used herein, “biogas” refers to a gaseous material comprising methane containing carbon and/or hydrogen that is derived from bio-based resources. A biogas recovered from biomass processing may also comprise CO₂. In one aspect, biogas comprises methane and CO₂ in a molar ratio ranging from 80:20 to 20:80, or 70:30 to 30:70, or 60:40 to 40:60, or 50:50.

As used herein, “biomass” refers to solid or liquid material of biological origin or from municipal solid or liquid wastes, from agricultural solid and liquid wastes, from forestry products, or from any other natural products or waste such as seaweed or sea plants, including on-purpose agricultural products made for gasification, much of which are derived ultimately from materials having a biological origin.

As used herein, “syngas” or, in the alternative, “synthesis gas” refers to a mixture of H₂ and CO in various ratios. Syngas may also contain one or more of CO₂, CH₄, and H₂O.

As used herein, “oxygenate” refers to hydrocarbons containing oxygen. Examples include alcohols, such as methanol (MeOH) and dimethyl ether (DME). Biooxygenate, biomethanol and biodimethyl ether are these materials derived from bio-based synthesis gas.

As used herein, bio-based CO refers to CO containing carbon that is sourced from renewable sources, from biological sources, or from carbon capture process involving capture of CO and CO₂ from the atmosphere, from flue gas and the like.

As used herein, the terms “reaction zone temperature” and “catalyst temperature” refer to the average catalyst bed temperature during the catalytic reaction process. In one aspect, the catalyst temperature is a numerical average of the temperature of the operating catalyst bed at the feed inlet and the temperature of the operating catalyst bed at the product outlet.

Unless otherwise specified, pressures reported in psi units are intended to indicate gauge pressure, or psig (pounds per square inch gauge).

As used herein, the term “molecular sieve” is a crystalline substance with pores of molecular dimensions which permit the passage of molecules below a certain size. It is commonly used as a commercial adsorbent and catalyst. Exemplary molecular sieves include phosphate molecular sieves (comprising silicon, aluminum, phosphorous, oxygen); and zeolites (comprising silicon, aluminum, and oxygen). Non-limiting examples of zeolitic molecular sieves include Beta zeolite, Y-zeolite, SSZ-13, or ZSM-5. In the context of a catalyst particle, “non zeolitic” refers to a catalyst containing no zeolites or phosphate molecular sieves. In the context of a catalyst bed, “non-zeolitic” refers to a bed of catalyst particles containing no zeolites or phosphate molecular sieves.

The gaseous feed to the reforming reaction zone comprises methane, in some cases with decreasing amounts of C2+ higher hydrocarbons, recovered from a source of biogas. Biogas that is generated for use according to the present disclosure contains biomethane or methane which is derived in part or in whole from a bio-based resource. Exemplary sources of bio-based methane include (i) methane obtained from anaerobic bacterial digestion of agricultural waste, municipal biowastes or from wastewater treatment, (ii) gaseous products of biomass conversion (e.g., composting, biomass gasification, pyrolysis, or hydro-pyrolysis, such as in the case of supercritical water gasification of biomass), (iii) landfill gases, or (iv) gaseous products of the electrochemical reduction of carbon dioxide. Carbon from bio-based carbon sources is termed “bio-based carbon”.

In some aspects, hydrogen may be added in the process to, for example, adjust the H₂/(CO+CO₂) content of a synthesis gas feed. Suitable hydrogen sources may include petroleum processing. Alternatively, bio-based hydrogen may be sourced from renewable sources, from biological sources, from electrolysis of water using solar, wind, wave, or other renewable energy sources or from naturally occurring geological hydrogen (commonly referred to as natural, gold or white hydrogen) or from nuclear powered water electrolysis (commonly referred to as pink hydrogen). Bio-based hydrogen is not, in general, formed by reactions of carbon compounds by steam reforming of methane.

Biogas may also contain CO₂, CO, ethane, water vapor, and nitrogen, depending on the specific process from which the biogas is generated. Raw (untreated) biogas may be passed to a reforming process without further treatment. Alternatively, non-methane components may be removed, either in part or in whole, and the treated biogas passed to the reformer for conversion into bio-based synthesis gas. Water that is present in the raw biogas may be condensed and removed from the biogas, using, for example, a water knockout pot for the two-phase separation. Non-hydrocarbon compounds (such as sulfur-, or nitrogen-, or acid-containing compounds) that are present in the raw biogas are removed to low levels, and often to ppm levels, using, for example, one or more of aqueous washing, alkanolamine absorption, molecular sieve adsorption, selective catalytic oxidation, and hydrodesulfurization.

Carbon dioxide may be removed from the raw biogas in combination with sulfur removal. Additional CO₂ may be removed by membrane separation, by cryogenic distillation or by aqueous absorption, which includes contacting the biogas with water or caustic solutions to dissolve CO₂, separating the water/CO₂ mixture, removing the CO₂ from the mixture by increasing the temperature and/or decreasing the pressure of the mixture, and recycling the water. CO₂ may also be removed in part by aqueous absorption into the water that is condensed and removed from the biogas. In some embodiments, at least a portion of one or more of CO₂, CO and water vapor may be retained in the treated biogas feed to maintain the desired H₂/(CO+CO₂) ratio of the bio-based synthesis gas exiting the reformer.

Carbon dioxide and/or water may also be added to the biogas feed from an external source to the reformer to control the H₂/(CO+CO₂) ratio in the bio-based synthesis gas produced in the reformer. In embodiments, carbon in the added carbon dioxide is from a bio-based resource, with the addition of carbon from a bio-based resource controlled to maintain a biogas carbon content of, for example, at least about 70 weight % that is bio-based carbon not derived from petroleum.

Recycle gas comprising H₂, CO, CO₂ and optionally methane and traces of C2+ hydrocarbons may also be added to the biogas, prior to passing the biogas as feed to the reforming reaction zone.

Bio-based synthesis gas comprising H₂ and CO may be produced by contacting a biogas comprising biomethane with an oxidizing gas selected from O₂, CO₂ and H₂O or combinations thereof at reforming reaction conditions in a reforming reaction zone to produce a bio-based synthesis gas comprising H₂ and CO.

Bio-based synthesis gas may be produced by biomass gasification, involving contacting biomass with some combination of air, oxygen, and/or steam at elevated temperatures. Fluidized-bed, fixed-bed or indirect heated gasifiers may be used. Varying steam to oxygen ratio input is a way to adjust the H₂/(CO+CO₂) ratio to match synthesis gas requirements. Gasifier temperatures may be between about 1,000° C. and about 1,300° C. or higher in some operations.

In another aspect, syngas (or alternatively bio-based synthesis gas) may be produced in a methane reformer involving steam reforming, autothermal reforming or partial oxidation to convert methane to hydrogen and carbon oxide gases. Either a fired biogas reformer or an electrical biogas reformer may be used. Reforming conditions include pressures between about 200 psi and about 600 psi (14-40 bar) and outlet temperatures between about 815° C. and about 925° C. Because the catalyst is sensitive to sulfur, the sulfur content of the biogas must be reduced to less than 10 ppm, preferably less than 1 ppm.

In steam methane reforming, steam reacts with methane as follows:

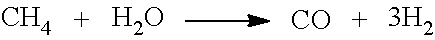

In the absence of steam, dry reforming proceeds as follows:

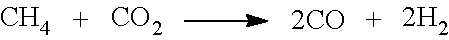

The reactants in the reforming reaction zone may also be converted by a water gas shift (WGS) reaction over the metal reforming catalysts (e.g., shaped nickel alumina catalysts):

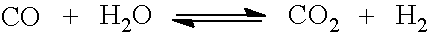

Therefore, adjusting the amount of CO₂ and H₂O added to the methane reforming reaction zone feed is useful for controlling the H₂/(CO+CO₂) ratio of the syngas generated during reforming. The composition of syngas generated in the reforming reaction zone, and in particular the H₂/(CO+CO₂) ratio in the syngas, may be controlled for efficient downstream conversion of the syngas to LPG. When the ratio is too low CO conversion is reduced. When it is too high large quantities of H₂ must be recycled. Synthesis of oxygenates in the oxygenate synthesis zone generally proceeds with a H₂/(CO+CO₂) molar ratio in the oxygenate synthesis zone feed in a range between 1 and 4 (e.g., in a range between 2 and 3). When the feed contains less than or equal to 1 mol % CO₂, the H₂/(CO+CO₂) ratio may be in a range between 2.25 and 2.45. When the feed contains more than 1 mol % CO₂ the H₂/(CO+CO₂) ratio may be in the range of 2.2 to 2.5.

For managing control of environmental emissions from the process, additional CO₂ may be added to the gaseous feed to the reforming reaction zone. In embodiments, the CO₂ used in the reformer is recovered either from the biogas generation reactor, from the recycle of unreacted products from the process, or from both. In particular, biogas generated from biomass includes CO₂ that may be removed from the biogas as a pure CO₂ product, making it highly suitable for blending into the blended synthesis gas feed to the oxygenate synthesis zone. Thus, in some cases, the synthesis gas feed may further comprise CO₂, for example in an amount of at least about 5 mol % (e.g., between about 4-50 mol % or between about 7-25 mol % or between about 8-10 mol %).

Controlling for the amount of water supplied to the oxygenate synthesis zone may also influence the synthesis reactions in the oxygenate synthesis zone. In particular, the addition of steam to the reaction zone, and the reaction of the steam with CO by WGS that is generated in a reforming reaction step in the oxygenate synthesis zone increases the H₂/(CO+CO₂) in the reaction zone. Likewise, increasing the CO₂ introduced to the oxygenate synthesis zone decreases the H₂/(CO+CO₂) by RWGS (reverse WGS).

Methane reforming for generating synthesis gas is generally conducted with a methane rich feed, comprising little or no C2+ components. Processes using a biogas feedstock containing excess C2+ hydrocarbons may include a pre-reformer for converting the C2+ hydrocarbons to methane. In embodiments, a primary source of C2+ components in the biogas feedstock is the light fraction recycle from the product recovery step. Suitable pre-reforming systems are known and are readily available.

Fresh bio-based synthesis gas that is supplied to an LPG oxygenate synthesis zone includes the bio-based synthesis gas produced in the reforming reaction zone. Other suitable sources of bio-based synthesis gas include one or more recycle streams generated in the process. Additional CO and/or H₂, some or all of which may be bio-based CO and/or bio-based H₂ may be supplied from external sources.

The process, according to the present invention, is directed at least in part to producing an LPG-enriched gaseous effluent by a catalytic synthesis process. In one aspect, the method comprises synthesizing an LPG-enriched gaseous effluent by converting a blended bio-based synthesis gas in one or more catalytic reaction zones. The blended bio-based synthesis gas is prepared by blending treated synthesis gas recycle stream and fresh bio-based synthesis gas. In one aspect, the volume ratio of recycled syngas to fresh syngas may be between 1 and 15, or between 2 and 8.

In another aspect, the method is directed to processes in which a synthesis gas recycle stream contains sufficient unsaturated hydrocarbons, including olefins and aromatics, to cause the catalysts used in oxygenate synthesis to foul and prematurely lose catalytic activity. The method provides for processes to reduce the unsaturated hydrocarbons content of the synthesis gas recycle stream, and to provide a treated synthesis gas recycle stream having an unsaturated hydrocarbons content of less than 5 mol %, based on the total moles of treated synthesis gas recycle stream that is recycled.

The synthesis catalyst comprises at least one oxygenate synthesis catalyst for converting the bio-based synthesis gas into oxygenates such as methanol, and an oxygenate conversion catalyst for converting the oxygenates into hydrocarbons, including LPG.

In embodiments, the bio-based LPG may be synthesized from bio-based synthesis gas in a dual-stage synthesis process, comprising reacting the blended bio-based synthesis gas in an oxygenate synthesis zone containing an oxygenate synthesis catalyst and forming an oxygenated reaction product comprising oxygenates and unreacted bio-based synthesis gas, wherein the oxygenates include at least 50 mol % methanol; and reacting at least a portion of the oxygenated reaction product in an oxygenate conversion zone containing an oxygenate conversion catalyst and forming the LPG-enriched gaseous effluent. The two-stage catalyst system may be part of a multi-stage catalyst system, in which the two stages of the system are separate, or, if in a single reaction vessel, spaced apart by a spacer element, such as a heat exchange element.

Oxygenate-containing effluent from the oxygenate synthesis zone may be heated by heat exchange, and the heated effluent passed to the oxygenate conversion zone for conversion to hydrocarbons, including LPG.

In embodiments, the oxygenate synthesis zone may be configured and operated to produce an effluent stream rich in MeOH. The oxygenate synthesis reaction proceeds by contacting a syngas (e.g., a bio-based synthesis gas) with a non-zeolitic oxygenate synthesis catalyst at synthesis reaction conditions. Synthesis reaction conditions include a first reaction zone temperatures of between about 200° C. and about 400° C., or between about 220° C. and about 350° C., or even between about 240° C. and about 280° C. The inlet pressure is between about 250 psi and about 1500 psi, or between about 400 psi and about 800 psi, or between about 600 psi and about 750 psi. The oxygenate synthesis catalyst comprises one or more oxygenate synthesis-active metals selected from the group consisting of Cu, Zn, Zr, Al, Pt, Pd, and Cr. CuZnAlOx (with a Cu/Zn/Al molar ratio of around 6:3:1) and ZnCrAlOx (with a Zn/Cr/Al molar ratio of around 1:1:2) are two suitable examples of an oxygenate synthesis catalyst.

To facilitate MeOH production in the oxygenate synthesis zone, the reaction zone contains essentially no molecular sieve or zeolitic component. As used herein, the term “essentially no molecular sieve or zeolite component” is understood to mean that there is insufficient molecular sieve or zeolite component in the catalyst to have a measurable effect on the performance of the catalyst, and more particularly on the formation of dehydrated oxygenates, such as DME. With the use of an oxygenate synthesis catalyst having essentially no molecular sieve component, more than 50 mol %, or more than 75 mol %, or more than 90 mol %, or even more than 95 mol % of the oxygenates in the oxygenate synthesis reactor effluent is MeOH. In one aspect, with the combination of catalysts as described herein for producing LPG, the per-pass CO conversion around both reaction zones is between about 25% and about 45%. With the overall LPG synthesis steps and recycle of unreacted syngas, the overall CO conversion is greater than 50%, or greater than 75%, or greater than 90%.

Bio-based MeOH is an important commodity for use in a variety of applications. Accordingly, a fraction of the MeOH that is generated in the oxygenate synthesis zone, and in some cases a large fraction, may be removed from the process, and only a fraction of the synthesized MeOH passed to the oxygenate conversion zone for conversion to LPG.

Gaseous effluent comprising MeOH from the oxygenate synthesis zone is passed, in whole or in part, to the oxygenate conversion zone. When available, additional MeOH from an external source may be added to the oxygenate conversion zone feed, including additional bio-based MeOH. Likewise, ethanol, ethylene and propylene from microbial fermentation of a biomass substrate may further contribute to the production of bio-based LPG.

Alternatively, some of the oxygenates present in the synthesis reaction product may be removed from the product and purified for other uses before the remainder of the synthesis reaction product is passed to the oxygenate conversion zone.

Prior to flowing the synthesis reaction product to the oxygenate conversion zone, the reaction product may be preheated to match the oxygenate conversion zone operating temperature, including, for example, by heating the synthesis reaction product to a temperature between about 280° C. and about 500° C., or between about 300° C. and about 475° C. before being passed to the oxygenate conversion zone.

While the synthesis reaction product flowing to the oxygenate conversion zone comprises MeOH in varying amounts, the stream also includes the unreacted syngas components H₂, CO and CO₂, hydrocarbons, and byproduct water. Most of the components in the oxygenate synthesis zone effluent, except for H₂ and MeOH, are inert under oxygenate conversion reaction conditions. Any water, hydrocarbons, CO, and CO₂ present in the effluent stream will pass through the oxygenate conversion zone, undergoing few, if any, reactions that alter the nature of the inert materials. The entire effluent stream from the oxygenate synthesis zone may therefore be passed to the oxygenate conversion zone for converting oxygenates in the effluent stream to hydrocarbons, including LPG.

In some applications of the process of the disclosure, a portion of the inert materials included in the effluent are removed before the remaining effluent is passed to the oxygenate conversion zone. For example, the presence of one or more of the inert components, when passed to the oxygenate synthesis zone, may change the concentration of reactions, and thereby reduce the reaction rate of the synthesis reaction.

In embodiments, the process includes converting the oxygenates formed in the oxygenate synthesis zone into paraffinic hydrocarbons, including C3 and C4 paraffinic hydrocarbons. The conversion reactions include dehydration of the oxygenates and saturation of olefins formed during dehydration, while limiting water gas shift reactions that convert available carbon in the reacting mix into CO₂.

In one aspect, the oxygenate conversion catalyst in the oxygenate conversion zone comprises a molecular sieve or zeolite. Non-limiting molecular sieves that are suitable for oxygenate conversion to produce an LPG-enriched gaseous effluent include SSZ-13, SAPO-18, SAPO-34, beta zeolite, ZSM-5 and Y-zeolite. In an embodiment, the oxygenate conversion catalyst converts all the oxygenates such that they are at low or undetectable levels in the effluent. This simplifies recovery of the desired LPG product.

In another aspect, the oxygenate conversion catalyst comprises a small-pore molecular sieve. In one aspect, use of a small pore molecular sieve promotes the formation of LPG relative to C5+ hydrocarbons in the gaseous effluent. Small pore molecular sieves that are suitable for the process include those molecular sieves where the openings to the pores are limited to 8-rings at the largest. The classification of pore sizes in molecular sieves is set forth by R. M. Barrer in Zeolites, 40 Science and Technology, edited by F. R. Rodrigues, L. D. Rollman and C. Naccache, NATO ASI Series, 1984. Examples of small pore molecular sieves include: Chabazite, SSZ-13, SAPO-34, SSZ-39, MCM-35, EU-12, RHO, SAPO-18, SAPO-56.

SSZ-13 is a suitable small pore molecular sieve for use as a catalyst in the oxygenate conversion zone. SSZ-13 is a synthetic chabazite (CHA)-type aluminosilicate zeolite mineral in the ABC-6 family of zeolites. SSZ-13 has a topology similar to the mineral chabazite, but SSZ-13 has a high silica composition. The Si/Al ratio is >5. In another aspect, the molecular sieve or zeolite component of the oxygenate conversion zone catalyst is characterized by a SiO₂/Al₂O₃ molar ratio of less than 200, or in a range between 10-90, or in a range between 10-30.

A typical hydrocarbon distribution of the second effluent of the present disclosure is illustrated in Table 1. This product was recovered from gaseous effluent produced by reaction over a SSZ-13 molecular sieve catalyst at a reaction temperature of 410° C.

TABLE 1

| Hydrocarbon | Weight % |
|---|---|
| Methane | 12.48 |
| Ethane | 13.64 |
| Propane | 65.92 |
| i-Butane | 0.04 |
| n-Butane | 6.39 |
| i-Pentane | 0.69 |
| n-Pentane | 0.08 |
| 2-Methylpentane | 0.46 |
| 3-Methylpentane | 0.30 |
| Total | 100 |

In another embodiment, the second effluent contains unsaturates at 1-6 mol % or higher.

The conversion catalyst may be compounded in a particulate alumina matrix and employed as spheres or extrudates in the reaction zone, the particulates having a cross-sectional diameter between 1/32 inch to ¼ inch. The extrudates may be shaped into tri-lobed (or similar) form to provide better access to the internal portion of the extrudate while maintaining mechanical strength.

In another aspect, the oxygenate conversion catalyst contains few, if any, metal species that are active for catalyzing water gas shift reactions. Metals that contribute to water gas shift activity of the conversion catalyst includes Fe, Cu, Zn, Pt, and Pd. The oxygenate conversion catalyst in the present process contains less than 5 weight % of these metals, or less than 1 weight % of these metals, or less than 0.1 weight % of these metals, either alone or in combination. In embodiments, the oxygenate conversion catalyst contains essentially no water gas shift active metal component. As used herein, the term “essentially no water gas shift active metal component” is understood to mean that there is insufficient metal component in the catalyst to have a measurable effect on the performance of the catalyst, and more particularly on the WGS activity of the catalyst.

The oxygenate conversion reaction is generally conducted at a temperature between about 280° C. and about 500° C., or between about 300° C. and about 475° C. In another aspect, the temperature of the gaseous feed to the oxygenate conversion zone is at least 50° C. greater than the temperature of the gaseous feed to the oxygenate synthesis zone. The pressure may be the same for both reaction zones with allowance for some pressure drop between about the reactors. Thus, the oxygenate conversion zone may operate at a pressure between about 250 psi and about 1500 psi, or between about 400 psi and about 800 psi, or even between about 600 psi and about 750 psi.

The LPG-enriched gaseous effluent exiting the oxygenate conversion reactor comprises H₂O, H₂, CO, CO₂, LPG, inerts (such as N₂) and C2− and C5+ hydrocarbons. In one aspect, the LPG-enriched gaseous effluent comprises hydrocarbons that are enriched in C4− hydrocarbons, including LPG. In another aspect, the LPG-enriched gaseous effluent comprises greater than 40 weight % LPG, or greater than 50 weight % LPG, or greater than 60 weight % LPG, or greater than 70 weight % LPG, based on the total saturated hydrocarbon content of the LPG-enriched gaseous effluent. In another aspect, the LPG-enriched gaseous effluent comprises less than 25 weight % C5+ hydrocarbons, or less than 20 weight % C5+ hydrocarbons, or less than 15 weight % C5+ hydrocarbons, or less than 10 weight % C5+ hydrocarbons, or less than 5 weight % C5+ hydrocarbons, based on the total saturated hydrocarbon content of the LPG-enriched gaseous effluent.

Separation and recovery of LPG at high purity involves a separation sequence involving one or more separation steps. A dewatering step removes water from the LPG-enriched gaseous effluent. A liquid absorption solvent in, for example, a sponge oil absorption process may be employed for recovering most, if not all of the LPG contained in the effluent. A solid absorbent in, for example, a Pressure Swing Adsorption (i.e., PSA) process may be employed for removing C2− hydrocarbons from a recycle stream produced in the liquid absorption process. A fractional distillation process may be employed for the hydrocarbon/syngas separation. Additional fractional distillation steps may be employed to separate LPG from C2− and C5+ hydrocarbon components. Membrane separation may also be used to separate syngas components from the hydrocarbons.

Sponge Oil Absorption is a well-established commercial process that removes relatively heavier gaseous hydrocarbons (generally C3 and higher) from lighter gaseous hydrocarbons in a gas mixture by contacting the gas mixture with a hydrocarbon liquid (lean liquid) at elevated pressure and relatively lower temperature in an absorption zone. The heavier gaseous hydrocarbons preferentially absorb in the hydrocarbon liquid. The liquid hydrocarbon with the dissolved heavier gaseous hydrocarbons is referred to as a rich liquid. The rich liquid is then processed in a desorption zone at temperatures above those in the absorption zone and pressures below those in the absorption zone. The absorbed hydrocarbons are vaporized from the rich liquid, which is then recycled to the absorption zone as a lean liquid. In one aspect, the method includes removing at least a portion of hydrocarbons from the LPG-enriched gaseous effluent into the liquid absorption at a temperature of less than 50° C. and at a pressure between about 500 psi and about 1500 psi, between about 500 psi and about 1000 psi, between about 500 psi and about 800 psi, or between about 600 psi and about 800 psi. Desired properties of the hydrocarbon liquid include remaining a liquid at the conditions of the absorption zone and with no significant volatilization at the conditions of the desorption zone. In one aspect, the liquid absorbent may have a normal boiling point greater than 100° C. A variety of hydrocarbon liquids can be used including kerosene, diesel, jet fuel, heavy naphtha, n-hexadecane and light cycle oil. Non limiting examples are U.S. Pat. No. 2,930,752A, 3,477,946A, or 7,107,788B2, the contents of each of which are incorporated herein by reference.

Pressure Swing Adsorption (PSA) is a commercial process used to separate hydrogen and hydrocarbon gases. A pressure swing adsorption (PSA) module is suited for removing hydrocarbons, CO, and CO₂ from a gaseous stream by adsorption onto a selective adsorbent material (e.g., zeolites or activated carbon) while rejecting hydrogen. Use of a PSA module involves adsorbing the C2− hydrocarbons, CO, and CO₂ from at least a portion of the gaseous stream onto the solid adsorbent at an adsorption pressure above 400 psi, separating a light fraction 152 comprising the adsorbed C2− hydrocarbons, CO, and CO₂ from the solid absorbent at a pressure at least 25 psi below the adsorption pressure. Non-adsorbed H₂ is returned to the synthesis gas recycle stream 158 at a pressure above 400 psi. In context of this application, the adsorption unit should preferably adsorb hydrocarbons (e.g., C2− hydrocarbons) and not adsorb significant amounts of hydrogen. In this way the hydrocarbons are removed from the synthesis gas recycle stream and the hydrogen is returned to the synthesis gas recycle stream. The adsorbed hydrocarbons are desorbed and may be sent to a deethanizer. The C3+ hydrocarbons are recovered as a product, and the methane and ethane are used as fuel or feed to a biogas reformer. The adsorber will use a molecular sieve, commonly 5A molecular sieve, or a carbon molecular sieve. The adsorption unit will optionally include a dehydrator ahead of the adsorption unit. Exemplary dehydrators include glycol dehydrators and molecular sieve dehydrators.

In one aspect, the process provides a method for reducing the unsaturated hydrocarbons (primarily olefins and aromatics) that are generated in the oxygenate conversion zone, and to a lesser extent in the oxygenate synthesis zone. While a portion of the unsaturated hydrocarbons produced in the LPG synthesis reactions will be removed from the recycle along with the hydrocarbons, sufficient unsaturated hydrocarbons may remain in the synthesis gas recycle stream to cause accelerated fouling and deactivation of the oxygenate synthesis catalyst.

LPG synthesis is often accompanied by the synthesis of a small amount of unsaturated hydrocarbons (olefins and/or aromatics). Examples include ethylene, propylene, butene, pentene, benzene, toluene, and the like. These unsaturated hydrocarbons may be present in the LPG-enhanced gaseous effluent in amounts greater than 0.5 mol %, or greater than 1 mol %, and as high as 6 mol % or higher, based on the total moles of hydrocarbons in the LPG-enhanced gaseous effluent. A portion of these unsaturated hydrocarbons may not be captured and recovered but may instead be included in the synthesis gas recycle stream that is passed for further reaction in the oxygenate synthesis reactor.

In the process, unsaturated hydrocarbons that remain in the synthesis gas recycle stream are removed by one or more of hydrogenation, adsorption on a solid adsorbent, or absorption in a liquid, resulting in an unsaturated hydrocarbons content of a treated synthesis gas recycle stream of less than 5 mol %, or less than 2 mol %, or less than 1 mol %, or less than 0.1 mol %, or even less than 0.01 mol %, based on the treated synthesis gas recycle stream.

A treatment step for removing unsaturated hydrocarbons from the LPG-enriched gaseous effluent may process the effluent either before or after product water has been removed from the effluent (i.e., the effluent has been dewatered). Alternatively, a treatment step may remove unsaturated hydrocarbons from the synthesis gas recycle stream that remains after the hydrocarbons are separated from the LPG-enhanced gaseous effluent.

In the process, the unsaturated hydrocarbons are treated in one or more of a hydrogenation process, an adsorption process, or an absorption process.

In one aspect, the unsaturated hydrocarbon removal treatment step is a hydrogenation process. The unsaturated hydrocarbons in the reaction stream being treated are hydrogenated to form saturated hydrocarbons by reaction with hydrogen over a hydrogenation catalyst. Supplemental hydrogen can be added if necessary. The pressure may be within 100 psi of the pressure of the reaction stream at that point. In one aspect, the pressure is the same as the pressure of the reaction stream at that point. The temperature may be between 30° C. and 400° C., or between 80° C. and 300° C., or between 100° C. and 200° C., or the same temperature as the reaction stream at that point. The hydrogenation catalyst includes metals selected from the group consisting of Pt, Pd, Re, Rh, Ir, Re, Ni, Fe, Co, Mo, Al, and Si. The metals are preferably dispersed on a support. Supports are selected from the group consisting of alumina, silica, silica-alumina, clays and molecular sieves (either phosphate molecular sieves or non-phosphate molecular sieves). The products from the hydrogenation process can be included in the LPG product, included in the C5+ product or allowed to remain with the C2− hydrocarbons. Hydrogen used in the hydrogenation reaction may be produced in the process, e.g., in the reformer; it may be produced on-site in a separation reaction, e.g., dehydrogenation of a hydrogen-rich organic material, e.g., cyclohexane, methyl cyclohexane; or it may be produced by electrolysis or derived from another source, either internal or external to the process. Hydrogen produced from renewable sources and using renewable processes are desirable. In another embodiment, the unreacted H₂ contained in the effluent from the LPG synthesis reactor can be used.

In one aspect, the unsaturated hydrocarbon removal treatment step is an adsorption process. Examples of adsorbents include alumina, silica, clay, activated carbon, molecular sieves (either phosphate molecular sieves or non-phosphate molecular sieves) and metal-organic frameworks (MOFs). The adsorbent may contain a metal that coordinates with the unsaturated hydrocarbons. Examples of metals which coordinate are transition elements from period 4 of the Periodic Table (Sc to Cu), from period 5 of the Periodic Table (Y to Ag) and from period 6 of the Periodic Table (Lu to Au). Ag and Cu are preferred and should be in the +1 oxidation state. The adsorbent can also include an acid dispersed on the adsorbents listed above. Examples of acids include sulfuric acid and phosphoric acid. Phosphoric acid on the clay kieselguhr is one such example. The adsorbent may also include an oxidant such as potassium permanganate for increasing the effectiveness of the adsorbent for removing unsaturated hydrocarbons. The pressure and temperature of the adsorption process should be the same as the synthesis gas recycle stream at that point in the overall process. When the adsorbent is spent it is either replaced or the adsorbed unsaturated hydrocarbons removed by processes selected from oxidation to CO₂, steam stripping, stripping with a hot gas.

In one aspect, the unsaturated hydrocarbon removal treatment step is an absorption process. The pressure and temperature of the absorption process should be the same as the synthesis gas recycle stream at that point in the overall process. Examples of absorbents include sulfuric acid, phosphoric acid, and ionic liquids. The ionic liquid can be acidic such as a chloroaluminate ionic liquid. The ionic liquid can include metals which coordinate with unsaturated hydrocarbons. Examples of metals which coordinate are transition elements from period 4 of the Periodic Table (Sc to Cu), from period 5 of the Periodic Table (Y to Ag) and from period 6 of the Periodic Table (Lu to Au). Ag and Cu are preferred and should be in the +1 oxidation state. When the absorbent is spent it can be either discarded, oxidized to removal absorbed hydrocarbons and create acid precursors such as sulfur oxides and phosphorous oxides, or regenerated by hydrogenation.

In another aspect, the formation of unsaturated hydrocarbons in the LPG-enriched gaseous effluent may be controlled, at least in part, by selectively treating the oxygenate conversion catalyst. During conversion of syngas to LPG by use of a combination of a methanol synthesis catalyst and a methanol conversion catalyst, aromatics can be formed. When the desired product is LPG, these aromatics are unwanted and should be minimized. Aromatics can form within the pores of intermediate pore size (10- and 11-ring) zeolites and large pore size (12-ring and larger) zeolites. But when small pore zeolites (8- and 9-ring) zeolites are used the aromatics are formed primarily on exterior acid sites.

The present of aromatics is of particular concern when the overall LPG synthesis process uses a two-stage approach with an oxygenate synthesis zone (without any dehydration) followed by an oxygenate conversion zone. The aromatics can be present in the recycle gas and fed back to the oxygenate synthesis zone where they will adsorb and deactivate the oxygenate synthesis catalyst. The aromatics can be saturated to form C6+ cycloparaffins, but these too may adsorb and at least partially deactivate the oxygenate synthesis catalyst. Furthermore, formation of undesirable aromatics may be accompanied by a decrease in the yield of LPG. It is therefore desirable to reduce or eliminate the formation of aromatics on external acid sites of the small pore zeolites used as oxygenate conversion catalysts.

In one aspect, the oxygenate conversion catalyst in the oxygenate conversion zone comprises a zeolite or molecular sieve. Exemplary zeolites that are suitable for oxygenate conversion include SSZ-13, SAPO-18, SAPO-34, beta zeolite, ZSM-5 and Y-zeolite. In another aspect, the oxygenate conversion catalyst comprises a small-pore zeolite. Suitable examples of small pore zeolites include: Chabazite, SSZ-13, SAPO-34, SSZ-39, MCM-35, EU-12, RHO, SAPO-18, SAPO-56.

In one aspect, reducing the aromatic-forming tendency of a small pore zeolite includes selectively removing zeolitic surface acid sites. Exemplary methods for removing zeolitic surface acid sites include:
Treatment of zeolites with aqueous solutions of ammonium fluorosilicate (see, for example, Han, S., et. al., Selective Removal of Surface Acidity in ZSM-5 Zeolite Using (NH₄)₂SiF₆ Treatment, Journal of Catalysis, Volume 196, Issue 2, 10 Dec. 2000, Pages 375-378)
Use ammonium fluoroborate to substitute surface aluminum with boron as a relatively weaker acid (see, for example, Liu, Xinsheng & Xu, Ruren, Dealumination of zeolites using an aqueous solution of ammonium tetrafluoroborate, J Chem. Soc., Chem. Commun., 1989, 1837-1839).
Use H4EDTA to remove aluminum from the surface of zeolites (see, for example, Shyu, J. Z., et. Al., Surface analysis of dealuminated Y zeolites by ESCA, Applications of Surface Science, Volume 21, Issues 1-4, April 1985, Pages 297-303).
Use an acid treatment, such as HCl, HNO₃ and citric acid, to remove aluminum from zeolites (see, for example, Velichkina, L, et. al., Effect of Acid Treatment on the Properties of Zeolite Catalyst for Straight-Run Gasoline Upgrading, Catalysis Research 2021, Volume 1, Issue 4)
Use a vapor phase treatment with SiCl4 to remove surface acid sites from zeolites (see, for example, Anderson, J. R., et al., Surface Deacidification of ZSM5 by SiCl4 treatment: Assessment of Surface Specificity by Methylene Blue Adsorption, Catalyst Letters, volume 2, pages 279-285, (1989)).
Lanthanide Oxides can be used to deactivate surface acid sites of zeolites.
Surface acid sites can be removed by mechanical abrasion which removes the outermost portions of the zeolite crystals (see, for example, Inagaki, S., et. al., Mechanochemical Approach for Selective Deactivation of External Surface Acidity of ZSM-5 Zeolite Catalyst, ACS Appl. Mater Interfaces 2015, 7, 8, 4488-4493).

Exemplary methods for selectively deactivating zeolitic surface acid sites include:
Selective siliation using bulky silanes such as 3-aminopropyl-triethoxylsilane (see, for example, Ding, W., et. al., The Effects of Silanation of External Acid Sites on the Structure and Catalytic Behavior of Mo/H-ZSM5, Journal of Catalysis, Volume 206, Issue 1, 15 Feb. 2002, Pages 14-22.)
Selective siliation using bulky silanes such as diphenylmethylsilane (see, for example, Tago, T., et. al., Control of Acid-Site Location of MFI Zeolite by Catalytic Cracking of Silane and its Application to Olefin Synthesis from Acetone, Journal of Chemical Engineering of Japan, Vol. 42, Supplement 1, pp. s162-s167, 2009.)
Selective siliation using bulky silanes such as tetraethoxysilane and tetramethylsiloxane (see, for example, Weber, R. W., The Characterization and Elimination of the External Acidity of ZSM-5, University of Cape Town: Dissertation, Master of Science in Engineering, November, 1993.).

While it is important that the small pore zeolite has some acidity in its pores, the surface acidity is desirably at a low level. The Si/Al ratio is >5. In another aspect, the molecular sieve or zeolite component of the oxygenate conversion zone catalyst is characterized by a SiO₂/Al₂O₃ molar ratio of less than 200, or in a range between 10-90, or in a range between 10-30. In this regard the overall SiO₂/Al₂O₃ molar ratio may be 10 or greater, or 25 or greater or 50 or greater, or 100 or greater. The surface acidity as measured by the adsorption of methylene blue may be decreased by the treatments from the starting material by 25% or more, or 50% or more, or 75% or more, or 90% or more or eliminated entirely. The surface acidity measured by adsorption of methylene blue on the treated material may be 2 mmol/100 g or less, or 1 mmol/100 g or less, or 0.5 mmol/100 g or less, or 0.1 mmol/100 g or less when measured at a concentration of 2×10⁻⁴ mol/dm⁻¹.

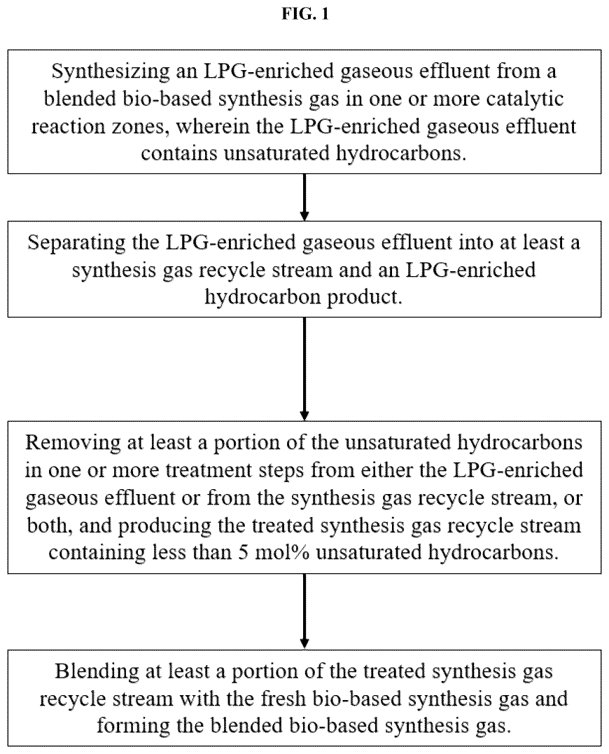

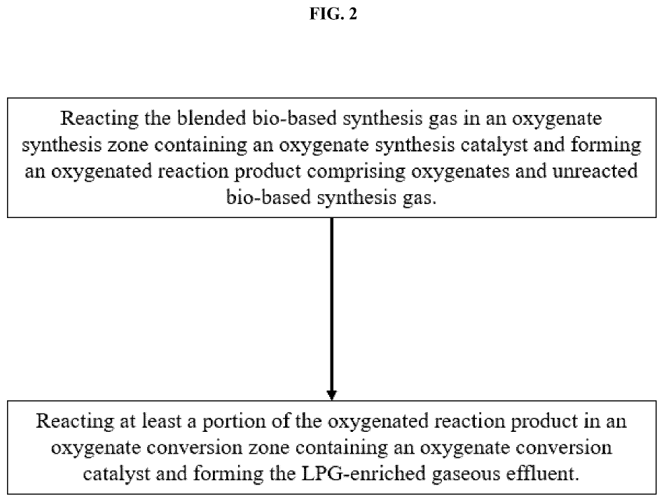

An exemplary embodiment of the process for utilizing recycle in the production of an LPG product may be understood by the following description, and in reference to the accompanying FIGS. 1-3. The FIGURES present illustrations of a process involving certain operational principles. To facilitate explanation and understanding, the FIGURES provides a simplified overview, and depicted elements are not necessarily drawn to scale. Valves, instrumentation, and other equipment and systems not essential to the understanding of the various aspects of the invention are not shown. As is readily apparent to one of skill in the art having knowledge of the present disclosure, processes for producing LPG via the reactions as disclosed herein, may have alternative configurations and elements that are governed by the specific operating objectives, but which alternatives are nonetheless within the scope of the invention.

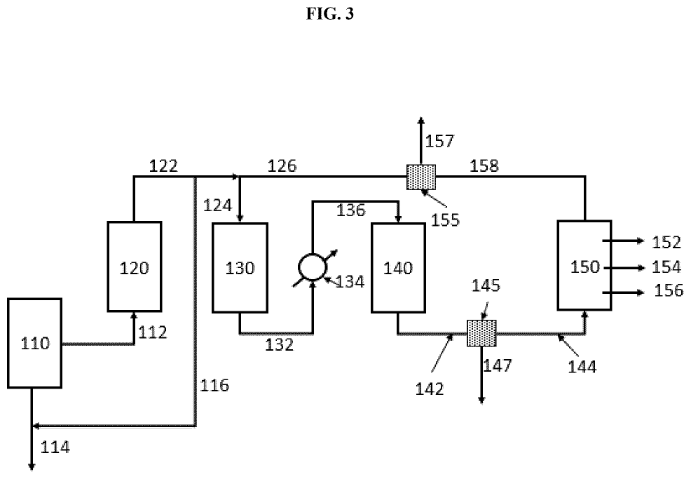

Referring to FIG. 3, biogas 112 may be produced from bio-based sources 110, including, for example, anaerobic bacterial digestion, composting, biomass gasification, pyrolysis or hydro-pyrolysis, landfill gases, or gaseous products of the electrochemical reduction of carbon dioxide. Biogas 112, optionally blended with light hydrocarbon streams from other sources, is passed to reforming reaction zone 120 for conversion to fresh bio-based synthesis gas 122 comprising CO, CO₂, H₂O, and H₂. Reforming reaction conditions may include pressures between about 200 psi and about 600 psi (14-40 bar) with outlet temperatures in the range of 815 to 925° C. The reforming reaction may take place over a shaped nickel alumina catalyst.

Contaminants in the biogas, including sulfur compounds and/or CO₂ in excess of that needed in downstream processing, may be removed from the biogas through vent stream 114. Biogas sulfur may be removed to low levels, and often to ppm levels, using, for example, one or more of aqueous washing, alkanolamine absorption, molecular sieve adsorption, selective catalytic oxidation, and hydrodesulfurization. Excess CO₂ may be removed from the biogas in combination with sulfur removal. Additional CO₂ may be removed by membrane separation, by cryogenic distillation or by aqueous absorption, which includes contacting the biogas with water to dissolve CO₂, separating the water/CO₂ mixture, removing the CO₂ from the mixture by increasing the temperature and/or decreasing the pressure of the mixture, and recycling the water. CO₂ may also be removed in part by aqueous absorption into the water that is condensed and removed from the biogas. In some aspects, CO₂ may also be removed through CO₂ recovery 116 from the fresh bio-based synthesis gas 122 for CO₂/CO ratio control.

The ratio of H₂/(CO+CO₂) in the fresh bio-based synthesis gas 122 is tailored to meet the requirements of downstream processing. Accordingly, the composition of the biogas feed to the reformer, including the amount of CO₂ and H₂O included in the biogas feed, may be modified to exploit reforming and/or water gas shift reactions to achieve the desired H₂/(CO+CO₂) composition of the fresh bio-based synthesis gas 122.

Blended bio-based synthesis gas 124 comprising fresh bio-based synthesis gas 122 and treated synthesis gas recycle stream 126 is passed to oxygenate synthesis zone 130, for synthesizing a gaseous oxygenate comprising methanol by reacting the blended bio-based synthesis gas 124 over a methanol synthesis catalyst in the oxygenate synthesis zone 130 to form a synthesis reaction product 132 that is enriched in MeOH. In one aspect, the oxygenate synthesis catalyst in the oxygenate synthesis zone comprises one or more oxygenate synthesis-active metals selected from the group consisting of Cu, Zn, Zr, Al, Pt, Pd, and Cr, with no molecular sieve component.

The oxygenate synthesis zone 130 may be operated at a temperature between about 220° C. and about 400° C. and at a pressure between about 250 psi and about 1500 psi. The synthesis reaction product 132 from the oxygenate synthesis zone 130, may be heated to a temperature between about 280° C. and about 500° C., or between about 300° C. and about 475° C. in a heating zone 134, and the heated reaction product 136 passed to the oxygenate conversion zone 140, in which oxygenates in the heated reaction product are converted to hydrocarbons, including LPG. The oxygenate conversion zone is operated at a pressure of between about 250 psi and about 1500 psi. In embodiments, the oxygenate conversion zone operates at a pressure of between about 500 psi and about 1500 psi or between about 600 psi and about 950 psi. These operating conditions contribute to reducing the olefin content of the LPG-enriched gaseous effluent 142 from the oxygenate conversion zone 140.

The pressures of the oxygenate synthesis zone and oxygenate conversion zone should be the same with small allowances for pressure drop between reactors (less than 50 psig).

In the process, the LPG-enriched gaseous effluent 142 may be dewatered and one or more LPG-enriched hydrocarbon products 152, 154, and 156 separated from a synthesis gas recycle stream 158. The LPG-enriched hydrocarbon product comprises bio-based LPG 154, light fraction, including C2− hydrocarbons 152, and C5+ hydrocarbons 156. The synthesis gas recycle stream comprises H₂, CO, and CO₂. Product recovery and unreacted gas recycle may take place in a one or more liquid and gaseous processing and separations. The LPG-enriched gaseous effluent 142, exiting oxygenate conversion zone 140 as a heated vapor, may first be cooled to condense at least a portion of the water vapor in a dewatering step (now shown), which may be removed for use elsewhere or for disposal.

In one aspect, the process is directed to treating the unsaturated hydrocarbons that may be formed in the oxygenate conversion zone and that remain in the synthesis gas recycle stream that is recycled to the oxygenate synthesis zone. The LPG-enriched gaseous effluent following the dewatering step may be treated in a first treatment step 145 to remove at least a portion of the unsaturated hydrocarbons contained in the LPG-enriched gaseous effluent.

Unsaturated hydrocarbons as byproducts of the LPG synthesis reactions may concentrate in the synthesis gas recycle if they are not removed, potentially resulting in synthesis catalyst fouling. The embodiments illustrated in FIG. 3 include several process locations where unsaturated hydrocarbons may be treated or otherwise removed to reduce the recycling of the unsaturated hydrocarbons. The choice of location for a particular application depends, at least in part, on the requirements of a specific practice of the process and on the stream being treated. In the example illustrated in FIG. 1, a treatment step 145 for removing unsaturated hydrocarbons may optionally be positioned for removing unsaturated hydrocarbons from the LPG-enriched gaseous effluent 142. Treated effluent 144 is then passed to separation process 150 for recovering hydrocarbons from the effluent.

Treatment step 145 may be selected from a hydrogenation method, an absorption method, and an adsorption method. In one aspect, treatment step 145 is a hydrogenation reaction process, in which the unsaturated hydrocarbons in the syngas recycle stream are hydrogenated to saturates by reaction with hydrogen in the synthesis gas recycle over a hydrogenation catalyst. Supplemental hydrogen may be added if necessary.

In another aspect, one or more of the separation processes may be an adsorption process, in which unsaturated hydrocarbon molecules are absorbed by a suitable absorbent, such as alumina, silica, clay, activated carbon, molecular sieves (either phosphate molecular sieves or non-phosphate molecular sieves) and metal-organic frameworks (MOFs). The adsorbent may contain a metal that coordinates with the unsaturated hydrocarbons. Examples of metals which coordinate are transition elements from period 4 of the Periodic Table (Sc to Cu), from period 5 of the Periodic Table (Y to Ag) and from period 6 of the Periodic Table (Lu to Au). Specific examples include Ag and Cu in the +1 oxidation state.

In another aspect, one or more of the separation processes for removing unsaturated hydrocarbons from the synthesis gas recycle stream may be an absorption process, using absorbents such as sulfuric acid, phosphoric acid, and ionic liquids. The ionic liquid may be acidic such as a chloroaluminate ionic liquid. The ionic liquid may include metals which coordinate with unsaturated hydrocarbons. Examples of metals which coordinate are transition elements from period 4 of the Periodic Table (Sc to Cu), from period 5 of the Periodic Table (Y to Ag) and from period 6 of the Periodic Table (Lu to Au). Specific examples include Ag and Cu in the +1 oxidation state. Unsaturated hydrocarbons 147 may be removed from the process for disposal, for use as fuel, or for use as feedstock to the reforming reaction zone 120.

In the example illustrated in FIG. 1, the LPG-enriched gaseous effluent 142, optionally with reduced unsaturated hydrocarbons content, is separated into at least a hydrocarbon-enriched fraction and a light gas fraction, in order to recover the bio-based LPG. The separation process 150 may include one or more separation steps for recovering bio-based LPG 154, light fraction 152, and C5+ hydrocarbons 156 from synthesis gas recycle stream 158.

In embodiments, the separation process 150 includes a sponge oil process, in which the hydrocarbons in the LPG-enhanced gaseous effluent are removed by absorption into a liquid absorbent.

The hydrocarbons may be removed from the liquid absorbent by fractionation. Additional fractionation steps serve to recover the bio-based LPG 154. When the hydrocarbons include C5+ hydrocarbons 156, these heavier hydrocarbons may be recovered as a separate product stream; likewise, the light fraction 152, that includes C2− hydrocarbons and unreacted syngas components, principally CO and CO₂. The light fraction 152 containing unreacted syngas components may be blended with the biogas 112 and passed to reforming reaction zone 120. Alternatively, the light fraction may be used as a fuel for internal or external use.

In embodiments, the separation process 150 includes a PSA process, in which the hydrocarbons in the LPG-enhanced gaseous effluent or the synthesis gas recycle stream are removed by adsorption in solid adsorbent adsorption unit into a liquid absorbent.

Hydrocarbon separation in separation process 150 may include two or more fractionators, each separating different hydrocarbon components distinguished by boiling point range. In this disclosure, the fractionators may be described by the term of art as a “deethanizer”, or as a “depropanizer”, or as a “debutanizer.”

Further in the example illustrated in FIG. 1, a treatment step 155 for removing unsaturated hydrocarbons may optionally be positioned for removing unsaturated hydrocarbons from synthesis gas recycle stream 158.

Treatment step 155 may be selected from a hydrogenation method, an absorption method, and an adsorption method. In one aspect, treatment step 155 is a hydrogenation reaction process, in which the unsaturated hydrocarbons in the syngas recycle stream are hydrogenated to saturates by reaction with hydrogen in the synthesis gas recycle over a hydrogenation catalyst. Supplemental hydrogen may be added if necessary.

In another aspect, one or more of the separation processes may be an adsorption process, in which unsaturated hydrocarbon molecules are absorbed by a suitable absorbent, such as alumina, silica, clay, activated carbon, molecular sieves (either phosphate molecular sieves or non-phosphate molecular sieves) and metal-organic frameworks (MOFs). The adsorbent may contain a metal that coordinates with the unsaturated hydrocarbons. Examples of metals which coordinate are transition elements from period 4 of the Periodic Table (Sc to Cu), from period 5 of the Periodic Table (Y to Ag) and from period 6 of the Periodic Table (Lu to Au). Specific examples include Ag and Cu in the +1 oxidation state.

In another aspect, one or more of the separation processes for removing unsaturated hydrocarbons from the synthesis gas recycle stream may be an absorption process, using absorbents such as sulfuric acid, phosphoric acid, and ionic liquids. The ionic liquid may be acidic such as a chloroaluminate ionic liquid. The ionic liquid may include metals which coordinate with unsaturated hydrocarbons. Examples of metals which coordinate are transition elements from period 4 of the Periodic Table (Sc to Cu), from period 5 of the Periodic Table (Y to Ag) and from period 6 of the Periodic Table (Lu to Au). Specific examples include Ag and Cu in the +1 oxidation state. Unsaturated hydrocarbons 157 may be removed from the process for disposal, for use as fuel, or for use as feedstock to the reforming reaction zone 120.

The unsaturated hydrocarbon content in the recycle stream may also be reduced by combining the oxygenate synthesis reaction and the hydrogenation reaction. In one aspect, the oxygenate synthesis catalyst is combined with a hydrogenation catalyst. In another aspect, the oxygenate synthesis catalyst and the hydrogenation catalyst are present in the reaction zone as separate particulate catalysts, blended uniformly in the reaction zone. In another aspect, the oxygenate synthesis catalyst is layered with the unsaturated hydrocarbon hydrogenation catalyst in alternating catalyst beds.

### EXAMPLE

The following non-limiting examples illustrate the content and technical solutions of the disclosure, but do not limit the scope of the invention.

### Example 1

The present process was modeled using AspenTech software to evaluate the losses of the synthesis gas components H₂, CO, and CO₂ from the process for various recycle recovery options. In each case, an LPG-enriched gaseous effluent was dewatered to form the dewatered gaseous effluent. Reaction conditions and process flows other than the composition of the recycle stream were kept constant. Data are summarized in Table 2.

Four processes were evaluated:
Run #1: The entire dewatered gaseous effluent is contacted in a liquid absorption solvent zone to remove hydrocarbons. A resulting hydrocarbon-depleted recycle stream is then contacted with a solid adsorbent to remove CO, CO₂, and remaining hydrocarbons.
Run #2: The entire dewatered gaseous effluent is contacted in a liquid absorption solvent zone to remove hydrocarbons. A 10% portion of the resulting hydrocarbon-depleted recycle stream is then removed as a purge from the recycle stream.
Run #3: The entire dewatered gaseous effluent is contacted in a liquid absorption solvent zone to remove hydrocarbons. A 10% portion of the resulting hydrocarbon-depleted recycle stream is then contacted with a solid adsorbent to remove CO, CO₂, and remaining hydrocarbons.
Run #4: A 40% portion of the dewatered gaseous effluent is contacted in a liquid absorption solvent zone to remove hydrocarbons. A 25% portion of the resulting hydrocarbon-depleted recycle stream from the absorption solvent zone is then contacted with a solid adsorbent to remove CO, CO₂, and remaining hydrocarbons.

The data in Table 2 tabulates the % losses of each synthesis gas component for each run, based on the total effluent flow leaving the conversion reaction zone.

TABLE 2
Gas Losses, % of flow to water knockout

| | Run #1 | Run #2 | Run #3 | Run #4 |
|---|---|---|---|---|
| H₂ | 12% | 10% | 1% | 1% |
| CO | 100% | 12% | 12% | 11% |
| CO₂ | 100% | 31% | 31% | 17% |

The data in Table 2 illustrate that losses of the unreacted synthesis gas components in the recycle are reduced by purging only a fraction of the recycle stream, rather than purging the entire recycle stream. Subjecting the purge to a PSA treatment and returning non-adsorbed H₂ has additional benefits of retaining CO, CO₂, and H₂ in the recycle stream. Surprisingly, the best result with respect to gas losses is realized when only a portion of the gaseous effluent is treated with the liquid absorption solvent, with only a portion of the hydrocarbon-depleted recycle stream from liquid absorption being treated using a PSA process.

### Example 2

In one aspect, the method for producing bio-based LPG includes operating under conditions to significantly reduce the amount of CO₂ that is generated by the method. Thus, reducing the reaction selectivity to form CO₂ is desirable. One reaction mechanism for producing CO₂ involves the water gas shift reaction. Water vapor added to the feed to the oxygenate synthesis stage, or water generated by the reactions occurring in the oxygenate synthesis reaction, are prone to react with CO in the reaction stage to form CO₂, rather than the CO being hydrogenated to the desired LPG product.

Run #5-7 are evaluated.

Run #5 involves converting CO in synthesis gas to LPG in a single stage reaction zone containing an oxygenate synthesis catalyst and an oxygenate conversion catalyst as a combined catalyst. During reaction under this reaction scheme, water is generated by a water gas shift reaction catalyzed by the metal components of the methanol synthesis catalyst. In this configuration, CO is converted to oxygen-free hydrocarbons, and for each mole of CO converted, one mole of water is formed. Water formed by reaction promotes sintering of the catalyst and the metals on this catalyst lead to formation of CO₂ by the water gas shift reaction.

Run #6 involves a two-stage reaction zone configuration, with a methanol synthesis catalyst and a methanol dehydration catalyst in the first stage, producing DME in the first stage effluent. The DME synthesized in the first stage is converted to LPG over a zeolite catalyst in the second stage. The two-stage configuration improves the per-pass conversion of carbon monoxide, but for each mole of CO converted to DME, 12 of a mole of water is formed in the first reactor.

Run #7, illustrates a method of the invention. Run #7 involves a two-stage reaction zone configuration, with a methanol synthesis catalyst in the first stage and a methanol conversion catalyst in the second stage. The first stage contains no molecular sieve component, and the product from the first stage reactor is almost exclusively methanol. Further, there is no significant formation of water per mole of carbon monoxide converted. The second stage contains a zeolite catalyst with no metal component that has water gas shift activity. In this configuration, CO conversion to hydrocarbons proceeds without the formation of water in excess of the water formed as a short-lived intermediate in methanol synthesis. This intermediate water is found to have little or no effect on catalyst sintering or in loss of CO by a water gas shift reaction.

The data tabulated in Table 3 illustrates the superior performance of the present method with respect to the formation of water during reaction in the oxygenate synthesis stage.

TABLE 3
Formation of Water in the Oxygenate Synthesis Reaction Zone

| | | | | Moles H₂O |
|---|---|---|---|---|
| | | | | formed per |
| | Reactor | Catalyst | | mole CO |
| | Configuration | First Stage | Second Stage | converted |
| Run | Single-Stage | Cu/ZnO/ | | 1 |
| #5 | | Al₂O₃ + | | |
| | | Beta zeolite | | |
| Run | Dual-Stage | Cu/ZnO/ | SSZ-13 | 0.5 |
| #6 | | Al₂O₃ + | | |
| | | ZSM-5 | | |
| Run | Dual-Stage | Cu/ZN/Al₂O₃ | SSZ-13 with no | Virtually little |
| #7 | | with no | WGS active | or no excess |
| | | molecular | metal | H2O produced. |
| | | sieve | component | |
| | | component | | |

As shown in Table 2, contacting 100% of the LPG-enriched gaseous effluent with both the sponge oil absorption and the PSA adsorption results in total loss of CO and CO₂. Contacting 100% of the gaseous effluent with the sponge oil absorption and then removing 10% of the gaseous recycle reduces the loss of H₂ to 10%, of CO to 12% and CO₂ to 31%. Loss of H₂ is reduced to 1% with a 10% purge of the recycle stream being directed to PSA adsorption. The lowest amount of H₂, CO, and CO₂ loss occurs when the sponge oil absorption treatment is applied to a 40% purge stream of the gaseous effluent, followed by a PSA adsorption treatment of 25% of the resulting recycle stream. This data clearly illustrates the benefit of using the sponge oil treatment and the PSA treatment of the gaseous recycle for recovering synthesis gas components from the recycle gas. The data also illustrates the additional benefit of treating only a fraction of the gaseous recycle using the two treatment steps.

### REFERENCE NUMBERS

| Reference No. | Description |
|---|---|
| 110 | biogas source |
| 112 | biogas |
| 114 | vent stream |
| 116 | CO₂ recovery |
| 120 | reforming reaction zone |
| 122 | fresh bio-based synthesis gas |
| 124 | blended bio-based synthesis gas |
| 126 | treated synthesis gas recycle stream |
| 130 | oxygenate synthesis zone |
| 132 | synthesis reaction product |
| 134 | heating zone |
| 136 | heated reaction product |
| 140 | Oxygenate conversion zone |
| 142 | LPG-enriched gaseous effluent |
| 144 | treated conversion product |
| 145 | first treatment step |
| 147 | unsaturated hydrocarbons |
| 150 | separation process |
| 152 | light fraction |
| 154 | bio-based LPG |
| 155 | second treatment step |
| 156 | C5+ hydrocarbons |
| 157 | unsaturated hydrocarbons |
| 158 | synthesis gas recycle stream |

1. A method for producing bio-based LPG, comprising:
a) synthesizing an LPG-enriched gaseous effluent from a blended bio-based synthesis gas in one or more catalytic reaction zones, wherein the blended bio-based synthesis gas comprises a treated synthesis gas recycle stream and a fresh bio-based synthesis gas, and wherein the LPG-enriched gaseous effluent contains unsaturated hydrocarbons;
b) separating the LPG-enriched gaseous effluent into a synthesis gas recycle stream and at least one LPG-enriched hydrocarbon product;
c) removing at least a portion of the unsaturated hydrocarbons contained in either the LPG-enriched gaseous effluent or the synthesis gas recycle stream, or both, in one or more treatment steps, and producing the treated synthesis gas recycle stream containing less than 5 mol % unsaturated hydrocarbons, based on the total moles of treated synthesis gas recycle stream that is recycled to the oxygenate synthesis zone; and
d) blending at least a portion of the treated synthesis gas recycle stream with the fresh bio-based synthesis gas and forming the blended bio-based synthesis gas of step a).

2. The method of claim 1, wherein the LPG-enriched hydrocarbon product comprises bio-based LPG, C2− hydrocarbons, and C5+ hydrocarbons.

3. The method of claim 1, wherein the fresh bio-based synthesis gas is prepared by contacting a biogas comprising biomethane with an oxidizing gas selected from O₂, CO₂ and H₂O or combinations thereof at reforming reaction conditions in a reforming reaction zone.

4. The method of claim 1, wherein the treated synthesis gas recycle stream contains less than 1 mol % unsaturated hydrocarbons, based on the total moles of treated synthesis gas recycle stream that is recycled to the oxygenate synthesis zone.

5. The method of claim 1, wherein the LPG-enriched gaseous effluent contains greater than 0.5 mol % unsaturated hydrocarbons, based on the total moles of hydrocarbons in the LPG-enhanced gaseous effluent.

6. The method of claim 1, wherein step a) of synthesizing an LPG-enriched gaseous effluent comprising steps of:
a) reacting the blended bio-based synthesis gas in an oxygenate synthesis zone containing an oxygenate synthesis catalyst and forming an oxygenated reaction product comprising oxygenates and unreacted bio-based synthesis gas, wherein the oxygenates include at least 50 mol % methanol; and
b) reacting at least a portion of the oxygenated reaction product in an oxygenate conversion zone containing an oxygenate conversion catalyst and forming the LPG-enriched gaseous effluent.

7. The method of claim 1, wherein the oxygenate synthesis catalyst comprises one or more methanol synthesis-active metals selected from the group consisting of Cu, Zn, Zr, Al, Pt, Pd, Rh, Ru, and Cr.

8. The method of claim 7, wherein the oxygenate synthesis catalyst contains essentially no molecular sieve or zeolitic component.

9. The method of claim 1, wherein the oxygenate conversion catalyst contains essentially no water gas shift active metal component, selected from the group consisting of Fe, Cu, Zn, Pt, and Pd.

10. The method of claim 6, wherein the oxygenate conversion catalyst comprises a zeolite having a SiO₂/Al₂O₃ molar ratio of less than 90.

11. The method of claim 6, wherein the oxygenate conversion catalyst comprises a small pore molecular sieve selected from Chabazite, SSZ-13, SAPO-34, SSZ-39, MCM-35, EU-12, RHO, SAPO-18, SAPO-56.

12. The method of claim 6, wherein the oxygenate conversion catalyst comprises SSZ-13.

13. The method of claim 6, wherein the oxygenate conversion catalyst comprises a small pore molecular sieve that has been prepared with a treated surface using a process selected from the group consisting of salination, surface abrasion, adsorption of lanthanide oxides, treatment with acids (HCL, HNO₃, citric), treatment with H₄EDTA, treatment with ammonium fluorosilicate, and treatment with ammonium fluoroborate.

14. The method of claim 13, wherein the small pore molecular sieve has a SiO₂/Al₂O₃ ratio in a range between 10-90 and a surface acidity measured by adsorption of methylene blue of 2 mmol/100 g or less.

15. The method of claim 1, wherein the LPG-enhanced gaseous effluent comprises greater than 40 weight % LPG, based on the total hydrocarbon content of the LPG-enhanced gaseous effluent.

16. The method of claim 1, wherein the LPG-enhanced gaseous effluent comprises less than 25 weight % C5+ hydrocarbons, based on the total hydrocarbon content of the LPG-enhanced gaseous effluent.

17. The method of claim 1, wherein the treatment step c) is a hydrogenation process comprising passing either the LPG-enriched effluent or the synthesis gas recycle stream over a metal-containing catalyst selected from the group consisting of Pt, Pd, Re, Rh, Ir, Re, Ni, Fe, Co, Mo, Al, and Si at a temperature between 30° and 400° C.

18. The method of claim 1, wherein the treatment step c) is an adsorption process comprising contacting either the LPG-enriched effluent or the synthesis gas recycle stream with an adsorbent selected from alumina, silica, clay, activated carbon, molecular sieves (either phosphate molecular sieves or non-phosphate molecular sieves) and metal-organic frameworks.

19. The method of claim 18, wherein the adsorbent contains one or more metals that coordinate with unsaturated hydrocarbons.

20. The method of claim 19, wherein the one or more metals that coordinate with unsaturated hydrocarbons is selected from copper and silver.

21. The method of claim 1, wherein the treatment step c) is an absorption process comprising contacting either the LPG-enriched effluent or the synthesis gas recycle stream with an adsorbent selected from sulfuric acid, phosphoric acid, and an ionic liquid.

22. The method of claim 21, wherein the ionic liquid contains one or more metals that coordinate with unsaturated hydrocarbons.

23. The method of claim 22, wherein the one or more metals that coordinate with unsaturated hydrocarbons is selected from copper and silver.

24. The method of claim 20, wherein the ionic liquid is a chloroaluminate ionic liquid.
